(19) United States
(12) Patent Application Publication
(10) Pub. No.: US 2014/0328951 A1
(43) Pub. Date: Nov. 6, 2014

(54) Composition for Preventing and Treating Vision Deterioration and Age-Related Macular Degeneration through Retinal Repair Using Ginseng/Red Ginseng Extracts and Ginsenoside

(71) Applicant: GBIOMIX. CO., LTD., Jeollabuk-do (KR)

(72) Inventors: Hyeon Jae Shim, Jeollabuk-do (KR); Yong Dol Shin, Jeollabuk-do (KR); Min Young Kang, Jeollabuk-do (KR); Jae Hwan Seok, Jeollabuk-do (KR); Hussain Ali, London (GB); Yun Hee Lee, Jeollabuk-do (KR); Cheul Mu Sim, Jeollabuk-do (KR)

(73) Assignee: GBIOMIX. CO., LTD., Jeollabuk-do (KR)

(21) Appl. No.: 14/350,180

(22) PCT Filed: Oct. 5, 2012
(86) PCT No.: PCT/KR2012/008107

§ 371 Date: Apr. 7, 2014

(30) Foreign Application Priority Data
Oct. 7, 2011 (KR) 10-2011-0102206
Oct. 10, 2011 (KR) 10-2011-0103258
Oct. 5, 2012 (KR) 10-2012-0110635

(51) Int. Cl. A61K 36/258; A61K 31/704

(52) U.S. Cl. CPC A61K 36/258; A61K 31/704
USPC 424/728; 514/26

(57) ABSTRACT

The present invention provides a composition comprising ginseng extract, red ginseng extract or ginsenosides as active ingredients for the prevention, slowed progression, and treatment of macular degeneration. More specifically it provides pharmaceutical composition to improve visual function by regenerating the transport characteristics of Bruch's membrane.

The present invention provides a composition comprising ginseng extract, red ginseng extract or ginsenosides as active ingredients to maintain and protect visual function in the normal population and restore deficits in visual function in the elderly population. More specifically it provides a composition to improve visual function by regenerating the transport characteristics of Bruch's membrane.

### TECHNICAL FIELD

The present invention provides a composition comprising ginseng extract, red ginseng extract or ginsenosides as active ingredients for the prevention, slowing progression and, treatment of macular degeneration. More specifically it provides pharmaceutical composition to improve visual function by regenerating the transport characteristics of Bruch's membrane of the eye.

The present invention provides composition comprising ginseng extract, red ginseng extract or ginsenosides as active ingredients to maintain and protect visual function in the normal population and restore deficits in visual function in the elderly population. More specifically it provides a composition to improve visual function by regenerating the transport characteristics of Bruch's membrane.

### BACKGROUND ART

Ginseng is one of the medicinal products which has been used traditionally in the treatment of various diseases in Asian countries such as China, Korea, Japan.

The main active ingredient of ginseng is ginseng saponin called ginsenosides and they have anti-aging, anti-inflammatory, and antioxidant activity in the central nervous, cardiovascular, and immune systems (Wu J Y, et al., J. Immunol 148: 1519-25, 1992; Lee F C., Facts about ginseng, the elixir of life. Hollyn International. New Jersey, 1992; Huang K C., The pharmacology of Chinese herbs. CRC Press. Florida, 1999), anti-diabetic activity (Chang H M., Pharmacology and application of Chinese material medica. Vol1. World Scientific. Singapore, 1986) and antitumor activity (Sato K, et al, Biol Pharm Bull 17:635-9, 1994; Mochizuki M, et It is known to have a variety of biological activities, Biol Pharm Bull 18:1197-1202, 1995).

More than 30 species of ginsenosides have been isolated and characterised. Ginsenosides are glycosides containing an aglycone with a dammarane skeleton and they can be divided into protopanaxadiol species (Rb1, Rb2, Rc, and Rd) and protopanaxatriol (Rg1 and Re).

Orally administered ginsenosides are metabolized by human intestinal microflora producing a variety of secondary products with biological activities (M Karikura, et al, Chem Pharm Bull 39:2357-61, 1991; Kanaoda M, et al., J. Tradit. Med. 11:241-5, 1994; Akao T, et al., Biol. Pharm. Bull. 21:245-9, 1998).

For example, 20-O-β-d-glucopyranosyl-20(S)-protopanaxadiol also known as IH-901 or compound K is an intestinal bacterial metabolite derived from protopanaxadiol-type saponins such as Rb1, Rb2 and Rc. (Hasegawa H, et al., Planta Medica 63:463-40, 1997; Tawab M A, et al., Drug Metab. Dispos. 31:1065-71, 2003) Ginsenosides Rh1 and F1 are derived from protopanaxatriol-type saponins, Rg1 and Re (Hasegawa H, et al., Planta Medica 62:453-7, 1996; Tawab M A, et al., Drug Metab Compound K, Rh1, and F1. Dispos. 31:1065-71, 2003).

These metabolites exhibit various physiological activities. Compound K is known to induce an anti-metastasis or anti-cancer effect by blocking tumor invasion or preventing chromosomal mutation and tumor formation (Wakabayashi C, et al., Oncol. Res. 9:411-7,1998; Lee S J, et al., Cancer Lett. 144:39-43, 1999).

Rh1 showed a cytotoxic effect on the growth of various cancer cells (Odashima S, et al, Cancer Res 45:2781-4, 1985; Ota T, et al., Cancer Res. 47:3863-7, 1987; Lee H Y, et al., Proc. 6th Int. Ginseng symp. Seoul 127-31, 1993), and anti-allergic, anti-inflammatory activity (Park E K, et al, Int Arch Allergy Immunol 133:113-120, 2004).

F1 significantly reduced ultraviolet-B-induced cell death and protected HaCaT cells from apoptosis caused by ultraviolet B irradiation. Ultraviolet-B-induced apoptosis is related with ginsenoside-F1-mediated inhibition of ultraviolet-B-induced down-regulation of Bcl-2 and Brn-3a expression (Lee E H, et al, J. Invest. Dermatol, 121:607-13, 2003).

Currently, age-related macular degeneration (AMD) remains the single largest cause of untreatable blindness in the elderly population. About 30 million people are affected worldwide, and every year an additional 500,000 people have severe blindness due to AMD. AMD accounts for 40% of people over 65 years of age who are registered as legally blind. In Korea, AMD is the main cause of blindness followed by glaucoma and diabetic retinopathy. The prevalence of AMD is expected to increase given the predicted increase in the elderly population worldwide. Recently the prevalence rates increase dramatically with age and earlier onset of the disease at age 40 is reported.

The major risk factor for the development of AMD is ageing. Although ageing changes occur throughout the fundus, they are more profound in the macular region that is responsible for central vision. AMD progresses with age, leading eventually to blindness. With the expansion of the aging population worldwide, the incidence of age-related macular degeneration is also expected to increase.

Age-related macular degeneration is an eye condition that leads to the deterioration of the center of the retina, called the macula, leading to loss of central vision. AMD may be manifested by symptoms such as blurriness, dark areas or distortion in their central vision, and at end stage, a permanent loss of central vision.

Photoreceptor cells are the light detectors of the retina and the subsequent transfer of information to the brain constitutes the process of vision. Photoreceptors are the most metabolically active cells in the body and therefore require efficient delivery of nutrients and removal of waste products. Since they also operate in an environment rich in polyunsaturated fatty acids, light and high oxygen tension, they inevitably undergo considerable free-radical mediated damage. Mechanisms exist to renew these damaged components by the continuous recycling of photoreceptor outer segments by the retinal pigment epithelium (RPE).

Photoreceptor and RPE cells obtain their metabolic needs from the adjacent choroidal blood circulation (FIG. 1). Blood borne nutrients are released from the capillaries of the choroidal blood vessels and must first cross a thin extracellular matrix called Bruch's membrane before reaching the RPE and photoreceptor cells. Small nutrients such as glucose, oxygen, amino acids, etc move across Bruch's by simple passive diffusion down concentration gradients. Vitamins, trace metals and lipids are bound to carrier proteins and diffuse across Bruch's as intact complexes and release their payloads on interaction with the RPE. Waste products from photoreceptors and the RPE move across Bruch's in the opposite direction to be removed into the choroidal circulation.

Ageing of Bruch's membrane results in increased thickness, deposition of lipids and proteolipid complexes, ‘debris’ discarded by the RPE, increased collagen cross-linking, denatured collagen, and glycosylation mediated cross-links associated with advanced protein and lipid glycation end-products (AGES & ALES). In addition, there is considerable damage of components of Bruch's from free-radical mechanisms.

Further complications arise from proteins that undergo dimerization and polymerisation and subsequent deposition within the matrix of Bruch's. For example, in the matrix metalloproteinase (MMP) system involved in the normal regeneration of the membrane (FIG. 2), monomers of pro-MMPs 2&9 polymerise to produce high molecular weight entities (HMW1 and HMW2) which then further aggregate with pro-MMP9 to produce the large macromolecular weight MMP complex termed ‘LMMC’. The deposition of these and other protein aggregates serves to ‘clog’ the membrane, diminishing its transport characteristics.

Thus, ageing of Bruch's is associated with diminishing transport across the membrane and hence a lowered capacity for delivery of nutrients and removal of waste products. Fluid transport decreases exponentially with age with a half-life of the decay process being 16 years, i.e., the capacity for fluid transport is halved every 16 years of life (FIG. 3a). The diffusional transport of protein-sized molecules also declines rapidly throughout the human lifespan (FIG. 4a). Thus the capacity for delivery of metabolites attached to carrier proteins such as vitamin A, trace metals and lipids is expected to diminish with age.

In the elderly, the transport pathways across Bruch's membrane are considerably reduced increasing the risk of metabolic insufficiency. Inefficient delivery of essential nutrients is expected to compromise the anti-oxidant machinery within the RPE and photoreceptor cells increasing the susceptibility for damage. The reduction in transport across Bruch's has two consequences. Firstly, diminished delivery results in retinal deficiency of essential metabolites despite normal plasma levels of these components. Secondly, metal carriers become trapped within Bruch's increasing the risk of further metal ion mediated free radical damage to the membrane. Similarly, diminished removal of waste products (in particular lipo-proteinaceous debris extruded by the RPE) leads to further ‘clogging’ of Bruch's membrane.

Clinically, the effects of ageing Bruch's in the elderly are noticed by diminished scotopic thresholds due to inefficient re-cycling of vitamin A. Currently, oral administration of vitamin A is prescribed with some authorities advocating supplementation with metals and anti-oxidants. There are two problems with such a strategy. Firstly, only a selective combination of nutrients can be supplemented and hence a deficiency in other essential nutrients remains. Secondly, metal supplements are likely to be deposited within Bruch's (since transport is restricted) increasing the risk of further damage. The ideal solution would be to increase the transport pathways through Bruch's so that all the normal components (present in plasma) can be transported and the waste products generated by photoreceptors and RPE can be removed.

In AMD, the ageing changes in Bruch's membrane described above are considerably exaggerated. Functional deterioration in transport processes progresses at a much faster rate (FIGS. 3a & 4a). Thus fluid transport across Bruch's reaches failure thresholds much earlier in life. On reaching the failure threshold, fluid can no longer be transported across Bruch's and accumulates beneath the RPE leading to an RPE detachment. This in itself can lead to photoreceptor degeneration because the diffusional distance for metabolites between the photoreceptors and Bruch's is considerably increased.

In AMD, the diffusional rates for protein carriers decline at a faster rate and therefore metabolic support is diminished. These changes cause metabolic insufficiency leading to greater production of ‘debris’ from the RPE. Such a constrained environment for exchange of nutrients and waste products causes a metabolic insult leading to the death of RPE and photoreceptor cells of the retina.

Mega doses of vitamin A have been used to address the deficiency but the regime cannot be sustained because of the toxic nature of the vitamin. The AREDS formulation has advocated the use of vitamin and mineral supplements but its usefulness remains undetermined. As indicated previously, the supplementation with metals may lead to further deposition of these toxic metals within Bruch's augmenting the underlying problems.

Potential treatments must improve the transportation pathways across Bruch's membrane. The proposed retinal regenerative ginseng therapy method aims to improve the transport properties of Bruch's membrane by shifting the transport decay curves described in FIGS. 3a & 4a upwards so that the metabolic insults due to compromised transport can be avoided in patients with AMD (FIGS. 3b & 4b).

### REFERENCES

[1] Birkedal-Hansen H, Moore W G, Bodden M K, Windsor L J, Birkendal-Hansen B, DeCarlo A, Engler J A. (1993) Matrix metalloproteinases: a review. Crit. Rev. Oral Biol. Med. 4:197-250.
[2] Handa J T, Verzijl N, Matsunaga H, Aotaki-Keen A, Lutty G A, to Koppele J M, Miyata T and Hjelmeland L M. Increase in the advanced glycation end-product pentosidine in Bruch's membrane with age. Invest. Ophthalmol. Vis. Sci. 1999; 40: 775-779.
[3] Holz F G, Sheraidah G S, Pauleikhoff D and Bird A C. Analysis of lipid deposits extracted from human macular and peripheral Bruch's membrane. Arch. Ophthalmol. 1994; 112: 402-406.
[4] Hussain A A, Lee Y, Zhang J J, Marshall J. (2011) Disturbed matrix metalloproteinase activity of Bruch's membrane in age-related macular degeneration (AMD). Invest. Ophthalmol. Vis. Sci. 52:4459-66.
[5] Hussain A A, Starita C, Hodgetts A, Marshall J. (2010) Macromolecular characteristics of ageing human Bruch's membrane: implications for age-related macular degeneration (AMD). Exp. Eye Res. 90:703-710.
[6] Hussain A A, Lee Y, Marshall J. (2010) High molecular weight gelatinase species of human Bruch's membrane: compositional analyses and age-related changes. Invest. Ophthalmol. Vis. Sci. 51:2363-71.
[7] Hussain A A, Starita C, and Marshall J. (2004) Chapter IV. Transport characteristics of ageing human Bruch's membrane: Implications for AMD. In: Focus on Macular Degeneration Research, (Editor 0. R. Ioseliani). Pages 59-113. Nova Science Publishers, Inc. New York.
[8] Hussain A A, Rowe L, Marshall J. (2002) Age-related alterations in the diffusional transport of amino acids across the human Bruch's-choroid complex. Journal of the Optical Society of America, A, Optics, Image Science, & Vision. 19(1): 166-72.
[9] Karwatowski W S S, Jefferies T E, Duance V C, Albon J, Bailey A J & Easty D L. Preparation of Bruch's membrane and analysis of the age related changes in the structural collagens. (1995) Brit. J. Ophthalmol. 79: 944-952.
[10] Kassof A, Kassoff J, Buehler J, et al., A randomized, placebo-controlled, clinical trial of high dose supplementation with vitamins C and E, beta carotene, and zinc for age-related macular degeneration and vision loss: AREDS report No. 8. Arch Ophthalmol. 2001; 119:1417-36.
[11] Kumar A, El-Osta A, Hussain A A, Marshall J. (2010) Increased sequestration of matrix metalloproteinases in ageing human Bruch's membrane: implications for ECM turnover. Invest. Ophthalmol. Vis. Sci. 51:2664-70.
[12] Moore D J and Clover G M. The effect of age on the macromolecular permeability of human Bruch's membrane. Invest. Ophthalmol. Vis. Sci. 2001; 42: 2970-2975.
[13] Moore D J, Hussain A A, Marshall J. (1995). Age-related variation in the hydraulic conductivity of Bruch's membrane. Invest. Ophthalmol. Vis. Sci. 36(7): 1290-7.
[14] Owsley C, Jackson G R, White M, Feist R and Edwards D. Delays in rod mediated dark adaptation in early age-related maculopathy. Ophthalmol. 2001; 108: 1196-1202.
[15] Owsley C, McGwin G, Jackson G R, Heinburger D C, Piyathilake C J, Klein R, White M F, Kallies K. Effect of short term, high-dose retinol on dark adaptation in age and age-related maculopathy. Invest. Ophthalmol. Vis. Sci. 2006. 47(4):1310-8.
[16] Ramratten R S, van der Schaft T L, Mooy C M, de Bruijn W C, Mulder P G H and de Jong P T V M. Morphometric analysis of Bruch's membrane, the choriocapillaris and the choroid in ageing. Invest. Ophthalmol. Vis. Sci. 1994; 35: 2857-2864.
[17] Starita C, Hussain A A, Pagliarini S, Marshall J. (1996) Hydrodynamics of ageing Bruch's membrane: implications for macular disease. Exp. Eye Res. 62(5): 565-72.
[18] Steinmetz R L, Haimovici R, Jubb C, Fitzke F W, Bird A. Symptomatic abnormalities of dark adaptation in patients with age-related Bruch's membrane change. Br. J. Ophthalmol. 1993; 77:549-554.

### DISCLOSURE

### Technical Problem

The objective of this invention is to provide a pharmaceutical composition for delaying, preventing or treating macular degeneration with ginseng, red ginseng extract and/or ginsenosides Rg1, Rb1 and compound K.

The objective of this invention is to provide a health food composition for delaying, preventing or treating macular degeneration with ginseng, red ginseng extract and/or ginsenosides Rg1, Rb1 and compound K.

The objective of this invention is to provide a composition comprising ginseng, red ginseng extract and/or ginsenosides Rg1, Rb1 and compound K to improve visual function by regenerating the transport characteristics of human Bruch's membrane.

The objective of this invention is to provide composition comprising ginseng, red ginseng extract and/or ginsenosides Rg1, Rb1 and compound K to address visual deficits in the normal elderly population and to maintain good visual function.

### Technical Solution

In order to accomplish the above object, the present invention provides a pharmaceutical composition for delaying, preventing or treating macular degeneration with ginseng, red ginseng extract and/or ginsenosides Rg1, Rb1 and compound K.

The objective of this invention is to provide a health food composition for delaying, preventing or treating macular degeneration with ginseng, red ginseng extract and/or ginsenosides Rg1, Rb1 and compound K.

The objective of this invention is to provide a composition comprising ginseng, red ginseng extract and/or ginsenosides Rg1, Rb1 and compound K to improve visual function by regenerating the transport characteristics of human Bruch's membrane.

The objective of this invention is to provide a composition comprising ginseng, red ginseng extract and/or ginsenosides Rg1, Rb1 and compound K to address visual deficits in the normal elderly population and to maintain good visual function.

### DESCRIPTION OF DRAWINGS

FIG. 1. Age-related changes in the structure and function of human Bruch's membrane.

FIG. 2. Ageing changes in the MMP pathway of human Bruch's membrane. (Hussain, A A, Lee, Y, Zhang, J J, Marshall, J (2011) Disturbed Matrix Metalloproteinase Activity of Bruch's membrane in Age-Related Macular Degeneration. Invest Ophthalmol Vis Sci 52(7): 4459-4466)

FIG. 3a. The time course of ageing changes in the fluid transport properties of Bruch's membrane in both the normal population and those with age-related macular disease.

FIG. 3b. The effects of the retinal regenerative ginseng therapy protocol (ginseng, red ginseng extract, and/or ginsenosides such as Rg1, Rb1 and/or compound K) to elevate the decaying fluid transport curves so that the likelihood of pathological intervention can be considerably delayed or eliminated.

FIG. 3c. Ginseng mediated improvement in the hydraulic conductivity of human Bruch's membrane.

FIG. 3d. Elevation of transport decay curves from the failure threshold following treatment with ginseng.

FIG. 3e. Improvement in hydraulic conductivity of human Bruch's membrane with commercially used ginseng products. (1,2: Cheonjiyang, 3: Cheong-Kwan-Jang, 4:Geumsan-Korean red ginseng gold)

FIG. 4a. The time course of ageing changes in the diffusional properties of Bruch's membrane in both the normal population and those with age-related macular disease.

FIG. 4b. The effects of the retinal regenerative ginseng therapy protocol (ginseng, red ginseng extract, and/or ginsenosides such as Rg1, Rb1 and/or compound K) to elevate the decaying diffusional curves so that the likelihood of pathological intervention can be considerably delayed or eliminated.

FIG. 4c. Ginseng mediated improvement in the diffusional status of human Bruch's membrane.

FIG. 4d. Elevation of diffusional decay curves from the failure threshold following treatment with ginseng.

FIG. 5a. Release of bound and trapped MMP enzymes from Bruch's membrane after perfusion with ginseng.

FIG. 5b. Release of bound and trapped proteins from Bruch's membrane after perfusion with ginseng.

FIG. 6. Removal of lipid components from Bruch's membrane after treatment with ginseng.

FIG. 7. The effects of ginseng and its components and derivatives (Rg1, Rb1, Compound K) on the release of MMP species from pig RPE cells.

FIG. 8. Fractions of ginseng

### BEST MODE

The invention utilises Korean Red Ginseng (KRG), its individual components, and transformed products (e.g., Compound K) to increase the transport properties of human Bruch's membrane. KRG mediated improvement in the functional properties of Bruch's membrane can address (a) the retinal nutritional deficiencies associated with the elderly and (b) visual deficits in general population and (c) serve as a prophylactic/preventive measure to slow or reverse the progression of degenerative disease associated with AMD. Underlying mechanisms of the mode of action of KRG appear to be complex with multiple targets. The impact of KRG on transport properties of Bruch's is detailed below.

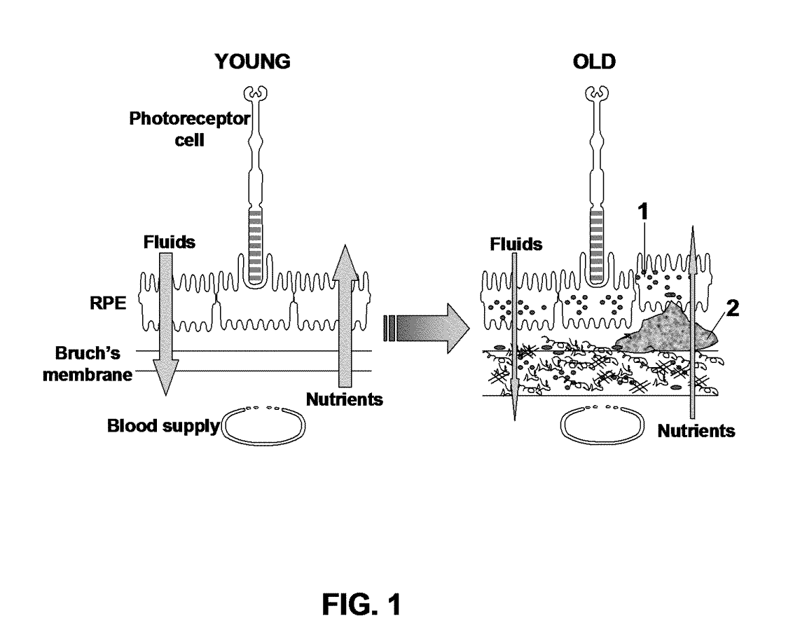

Photoreceptor and RPE cells obtain their metabolic needs from the adjacent choroidal blood circulation (FIG. 1). Blood borne nutrients are released from the capillaries of the choroidal blood vessels and must first cross a thin extracellular matrix called Bruch's membrane before reaching the RPE and photoreceptor cells. Small nutrients such as glucose, oxygen, amino acids, etc move across Bruch's by simple passive diffusion down concentration gradients. Vitamins, trace metals and lipids are bound to carrier proteins and diffuse across Bruch's as intact complexes and release their payloads on interaction with the RPE. Waste products from photoreceptors and the RPE move across Bruch's in the opposite direction to be removed into the choroidal circulation.

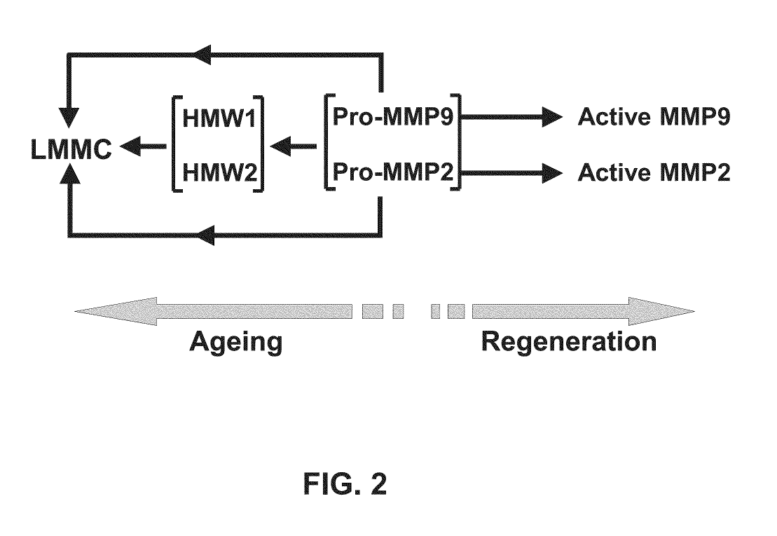

Ageing of Bruch's membrane results in increased thickness, deposition of lipids and proteolipid complexes, ‘debris’ discarded by the RPE, increased collagen cross-linking, denatured collagen, and glycosylation mediated cross-links associated with advanced protein and lipid glycation end-products (AGES & ALES). In addition, there is considerable damage to components of Bruch's from free-radical mechanisms. Further complications arise from proteins that undergo dimerization and polymerisation and subsequent deposition within the matrix of Bruch's. For example, in the matrix metalloproteinase (MMP) system involved in the normal regeneration of the membrane (FIG. 2), monomers of pro-MMPs 2&9 polymerise to produce high molecular weight entities (HMW1 and HMW2) which then further aggregate with pro-MMP9 to produce the large macromolecular weight MMP complex termed ‘LMMC’. The deposition of these and other protein aggregates serves to ‘clog’ the membrane, diminishing its transport characteristics.

Ageing results in thickening and deposition of lipo-proteinaceous material within Bruch's membrane. Material extruded by photoreceptors that cannot be digested by the RPE accumulates as lipofuscin granules (1 in FIG. 1). Other complex lipo-protein complexes termed drusen (2 in FIG. 1) accumulate on the surface of Bruch's. Such ageing changes diminish the transport processes across Bruch's making photoreceptors susceptible to metabolic insults. An early disturbance in visual function is detected in the elderly but in age-related macular disease, the ageing changes are severely advanced leading to the death of the RPE and photoreceptors, culminating in visual loss.

MMPs are proteolytic enzymes that are released as inactive pro-forms by the RPE into Bruch's membrane. The conversion of pro-MMPs to active MMPs 2&9 results in matrix degradation in the process of continuous regeneration of the membrane. Levels of these active forms were shown to be significantly reduced in Bruch's from AMD sufferers and may provide an explanation for the degenerative changes observed in the membrane. In the toxic environment of Bruch's, pro-MMPs polymerise to high molecular weight complexes called HMW1 and HMW2. These together with pro-MMPs aggregate to form even larger high molecular weight aggregates termed LMMC (FIG. 2). All these complexes become trapped or bound to the matrix diminishing the transport pathways through Bruch's. Pro and active MMPs also get trapped. These polymerisation and sequestration steps are not confined to just the MMPs since other proteins in the matrix are also expected to follow the same route. These alterations are detrimental to the functioning of Bruch's membrane.

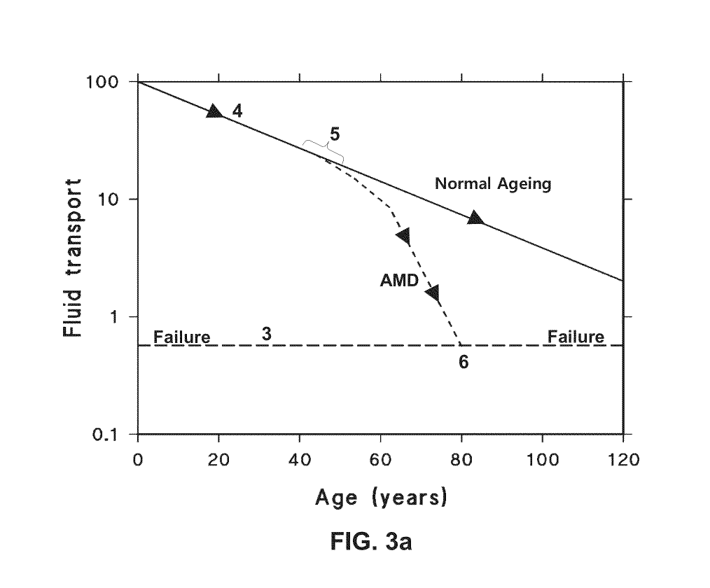

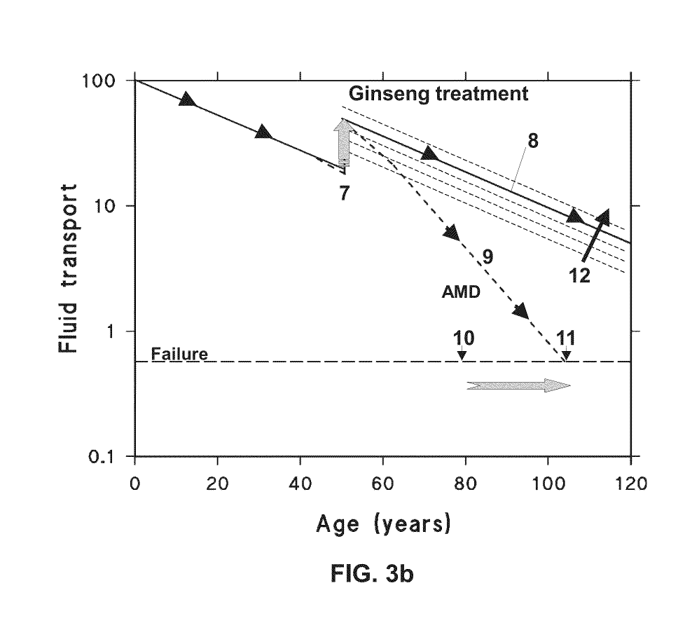

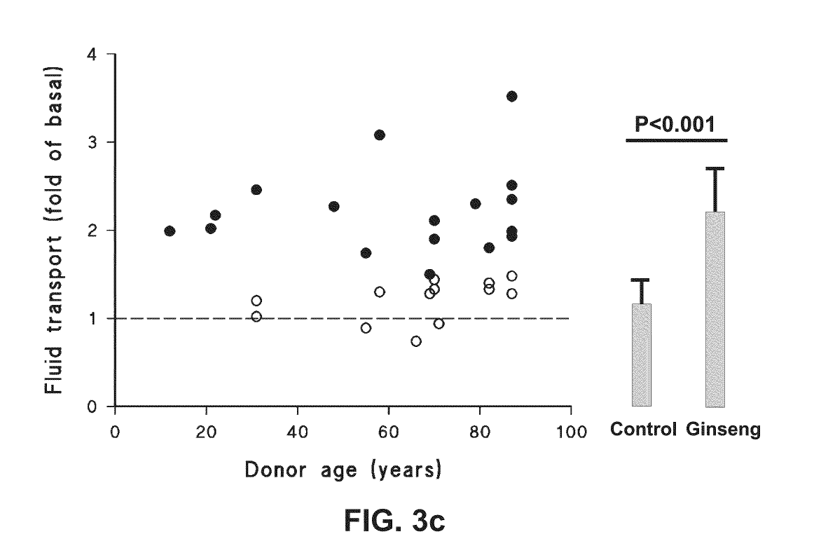

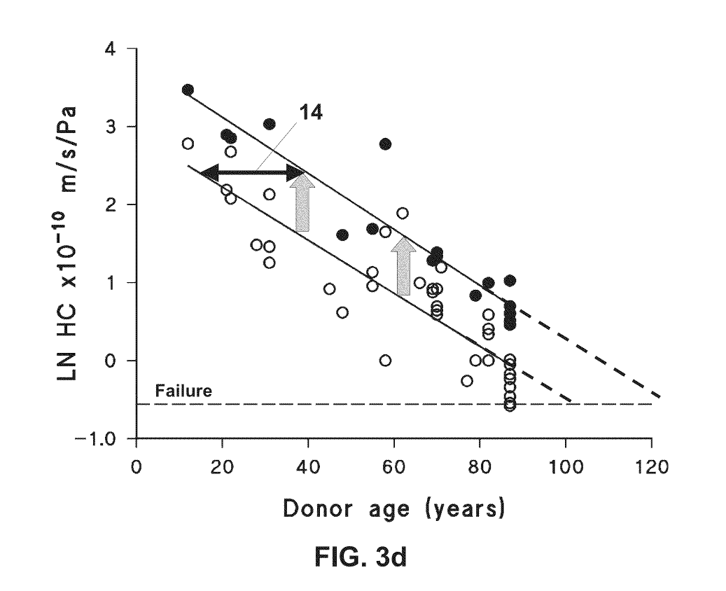

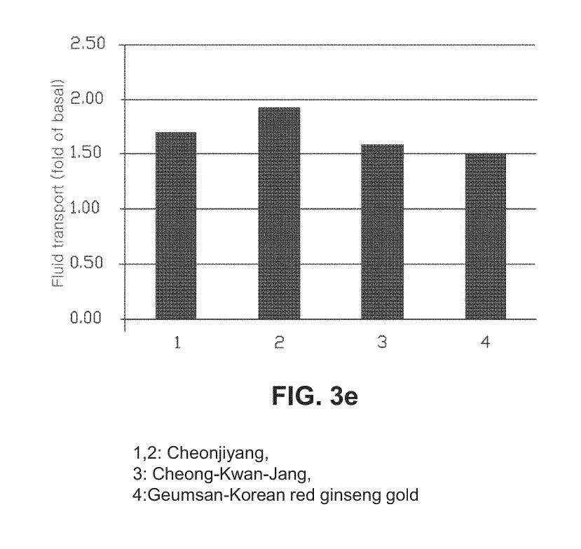

A minimum amount of hydraulic transport capacity is required in Bruch's to maintain the visual unit depicted by the failure line (3 in FIG. 3a). If transport rates fall below this line, then progression to pathology ensues. Ageing of Bruch's leads to an exponential decline in hydraulic conductivity, shown in the logarithmic plot as a straight line (4 in FIG. 3a). Generally, this line for the normal population does not meet the failure threshold within the human lifespan. However, normal elderly individuals approach this failure threshold and give rise to problems associated with abnormal night vision. In patients with AMD, the declining line begins to diverge around the ages of 40-50 years (5 in FIG. 3a) with a much steeper gradient, heading for the failure threshold (6 in FIG. 3a). The metabolic insult may then progress rapidly to the degenerative phase of the disease.

In order to avoid meeting the threshold lines, two possibilities exist. Either the thresholds are lowered or the declining hydraulic conductivity curves are elevated. Thresholds cannot be lowered because they represent the minimum transport capacity to maintain photoreceptor cells. However, it may be possible to elevate the declining curves with the retinal regenerative ginseng therapy protocol. If the retinal regenerative ginseng therapy protocol was instigated around the age of 50 years (7 in FIG. 3b), then the normal declining curve would be elevated, meeting the failure threshold well outside the normal human life span (8 in FIG. 3b). Similarly, the AMD declining curve (9 in FIG. 3b) would also be shifted, considerably delaying its meeting time with the failure threshold from point 10 (in FIG. 3b) to point 11 (in FIG. 3b). The degree of elevation of the decaying curves would be expected to be a function of retinal regenerative ginseng therapy dosage and is represented by the dashed lines (12 in FIG. 3b).

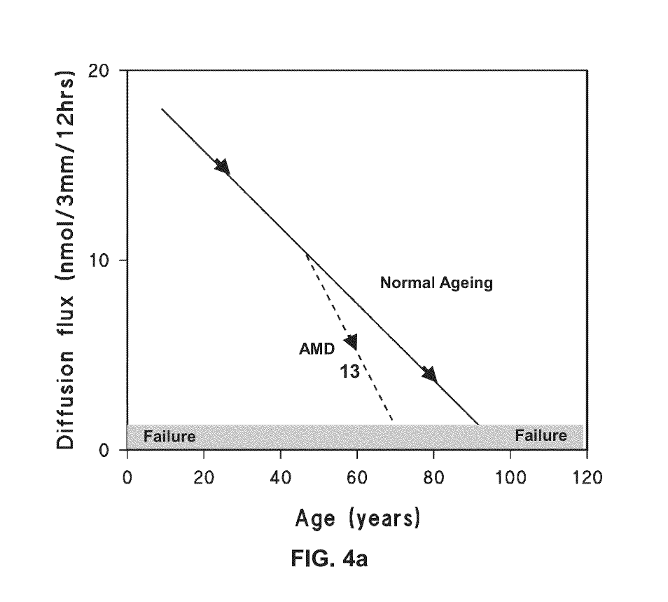

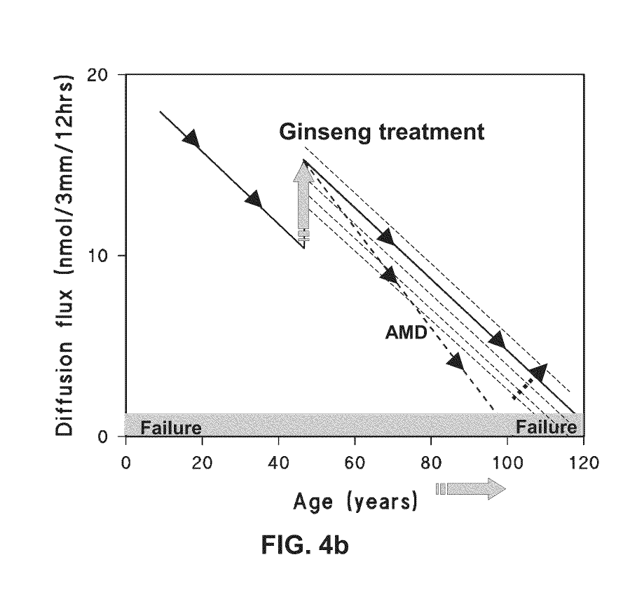

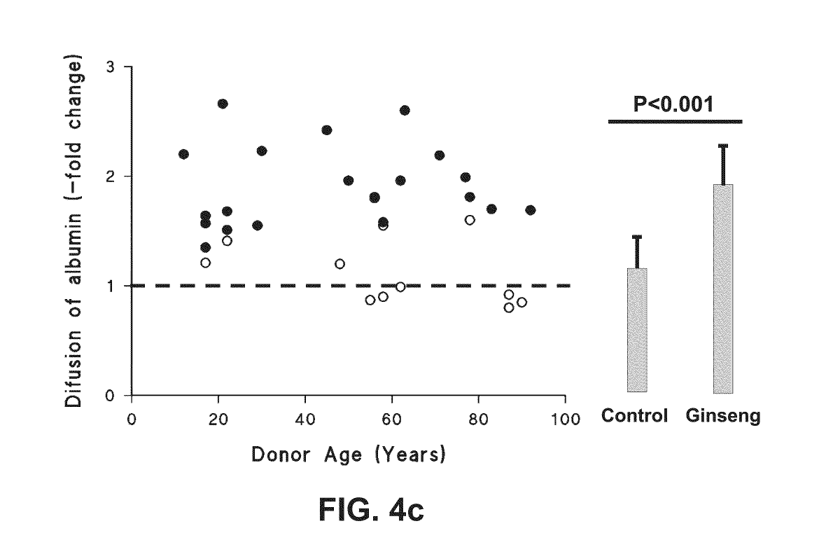

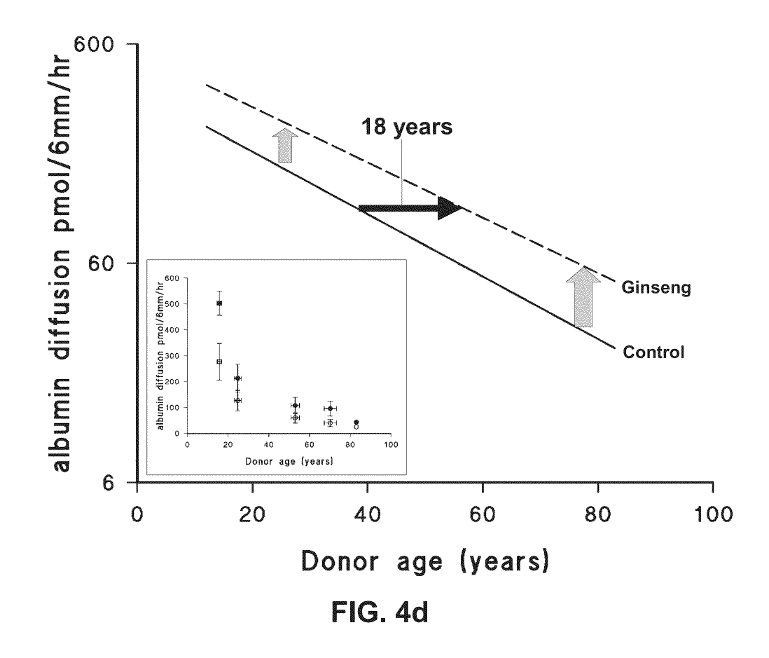

Diffusion status of Bruch's has previously been determined with respect to the movement of a dextran molecule of MW 22.4 kDa. The size of this molecule is similar to that of albumin or of protein carriers for metals. The diffusion of these macromolecules was shown to decrease linearly with age reaching failure thresholds between 80-100 years of age. Not surprisingly, many elderly people reach these thresholds leading to compromised problems in transport across Bruch's and often require considerable vitamin and mineral supplementation in their diets. In AMD, the decline has been shown to be much more steeper (13 in FIG. 4a) augmenting the pathological progression of the disease.

As with the hydraulic conductivity of FIG. 3b, retinal regenerative ginseng therapy may elevate the decaying lines for the diffusion process so that failure thresholds are not encountered within the human lifespan (FIG. 4b). In AMD subjects therefore, the point at which failure threshold is reached can be extended by retinal regenerative ginseng therapy protocol, protecting the RPE and photoreceptors from the metabolic insults that culminate in retinal degeneration.

Incubation with ginseng, red ginseng extract, and/or ginsenosides (Rg1, Rb1, Compound K) improved the hydraulic conductivity of donor samples. (FIG. 3c, FIG. 3d, Table2, and Table3). The hydraulic conductivities of basal and ginseng treated Bruch's preparations have been plotted on a semi-log scale in the figure. The best-fit non-linear regression lines show an exponential decay function for the basal level of conductivities, in keeping with previous publications. Incubation with ginseng, red ginseng extract, and/or ginsenosides (Rg1, Rb1, Compound K) elevated these lines towards improved hydraulic conductivities (FIG. 3c, FIG. 3d, Table2, and Table3). The shift in these lines (14 in FIG. 3d) was equivalent to a shift in hydraulic conductivities of about 25 years i.e., the hydraulic conductivities measured after ginseng treatment corresponded to conductivities expected of donors 25 years younger. The improved hydraulic conductivity means a delay of about 25 years before failure thresholds are met.

The age related decrease in the diffusion of dextrans across Bruch's has previously been shown to be linear. In our experiments with albumin, a globular protein, the decline in diffusion with age was observed to be exponential (inset FIG. 4d). For this reason, the non-linear regression relationship between diffusion and age has been plotted on a logarithmic plot for control and ginseng treated samples of Bruch's membrane. Ginseng incubations shifted the decay curves upwards with an average shift of 18 years (FIG. 4d)

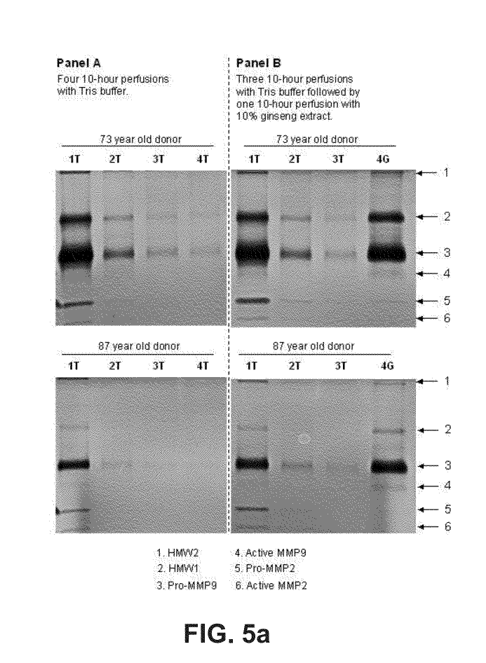

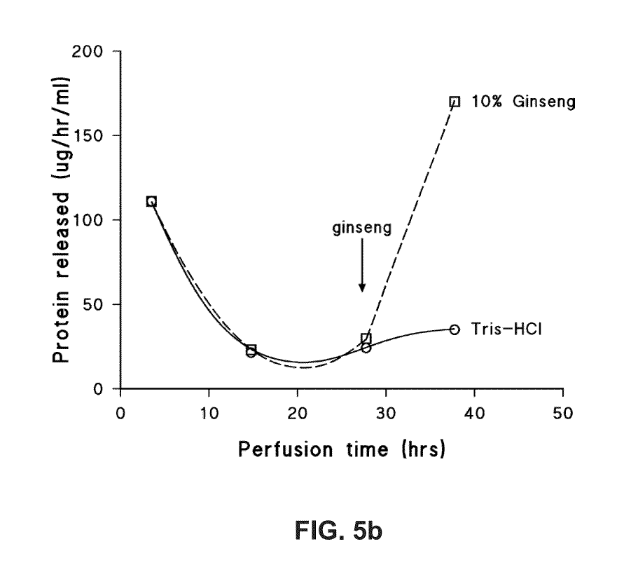

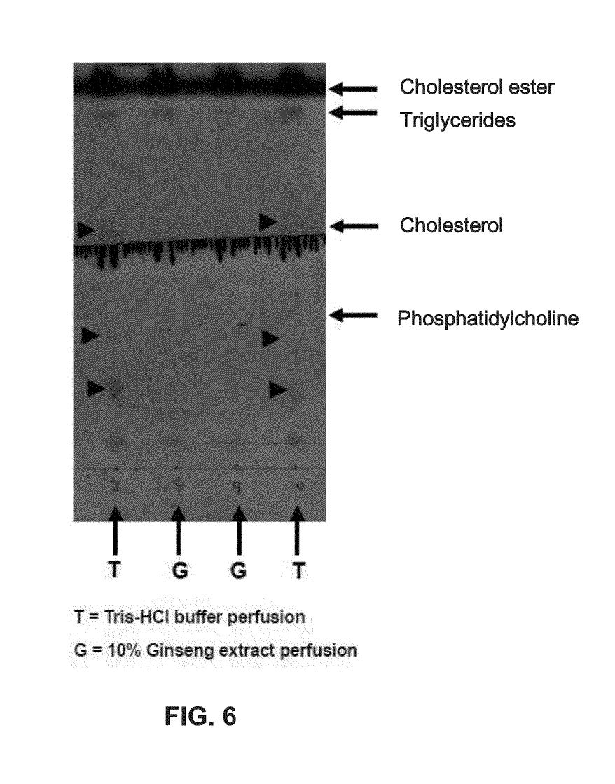

Ageing of Bruch's is associated with entrapment and binding of proteinaceous material to the matrix of the membrane, contributing to the loss in transport properties. Red ginseng extract released trapped and bound proteins from Bruch's membrane. KRG therefore, by releasing bound fractions from the matrix would contribute to improving the transport properties of the membrane (FIG. 5b). Thus, ginseng is capable of removing some of the lipid components of Bruch's membrane. This action of ginseng would assist in the regeneration of the membrane (FIG. 6). The removal of trapped or bound proteins and lipid by ginseng extract helps to regenerate Bruch's membrane (FIGS. 5b and 6).

The MMP species within Bruch's membrane exist in free and bound forms, the relative proportions remain unknown. However it is known that the bound fraction increases with age. An experiment was devised to assess if these bound MMP species could be released by ginseng perfusion (4G in FIG. 5a). Subsequent elution with 10% ginseng resulted in the release of large amounts of bound HMW2, HMW1 and pro-MMP9 species together with active enzymes. In the case of MMPs, the release of activated species would serve to degrade other abnormal components helping to regenerate the membrane.

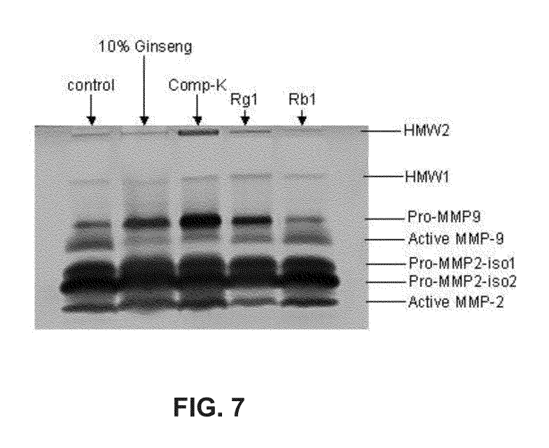

MMP species are released primarily by the RPE as inactive pro-enzymes. Activation requires the loss of a small peptide and is also mediated by the RPE. Activation of MMP9 occurs in response to cellular migration, damage and/or neovascular processes. Active MMP2 is a constitutive enzyme that is responsible for general turnover of Bruch's membrane. In control medium, both pro- and active MMP 2&9 species are released continuously from the RPE (FIG. 7). Incubation with 10% Ginseng extract resulted in increased release of pro-MMP9 but decreased release of active MMP-9. On the other hand, the release of both pro- and active MMP2 species was elevated. This effect of KRG is conducive for regenerating Bruch's membrane. Compound K consistently increased levels of Pro-MMPs 2&9 and active MMP2, again a very useful result for supplementing Ginseng extract with this compound. Ginsenoside Rb1 was effective in reducing the level of pro-MMP9 but without effect on the level of active MMP9. Rb1 however did increase levels of both pro- and active MMP2 species. Ginsenoside Rg1 was without effect on the release of MMPs from the RPE.

The ginseng mediated release of MMPs means that other trapped or bound proteins and lipids are also removed from ageing Bruch's membrane. The release of bound MMPs serves to improve and regenerate the function of the membrane (indicated by the increased hydraulic conductivity). Therefore, ginseng plays an important role for delaying, preventing or treating macular degeneration.

Therefore, ginseng, red ginseng extract and/or ginsenosides Rg1, Rb1 and compound K break up the polymerized protein complexes and release trapped/bound proteins and lipids that normally cause loss of function in Bruch's membrane. Removal of polymerized MMPs and release of active MMPs will restore enzyme function, so it helps to regenerate the membrane. Therefore hydraulic conductivity will be improved and serves to delay retinal ageing.

Ginseng/red ginseng extract and ginsenosides Rg1, Rb1 and compound K can be used as individual supplements or in combination. A more enhanced effect may be obtained with combination therapy with ginseng or ginseng extract, ginsenosides Rg1, Rb1 and compound K. Furthermore, this combination therapy of the present invention (ginseng/red ginseng extract and ginsenosides Rg1, Rb1 and compound K as active ingredients) can be extended to include other protective ingredients such as vitamins, minerals, and antioxidants to combat eye disease.

Ginseng/red ginseng extract and ginsenosides Rg1, Rb1 and compound K can be used as pre-treatment for patients undergoing stem or RPE cell transplantation as a possible treatment for age-related macular degeneration. The viability of the transplanted cells would be much enhanced if the transportation pathways of underlying Bruch's were improved prior to transplant enabling better attachment and survival of transplanted cells.

The present invention as described herein applies to ginseng and red ginseng for convenience. However, the present invention also incorporates various processed forms of ginseng, for example, raw ginseng, fine root, white ginseng, taekuksam, black ginseng, dextrinized ginseng, enzymatic treated ginseng, fermented ginseng, red ginseng, and fermented red ginseng on the scope of the present invention. The need for wider inclusion of various forms of ginseng would be obvious to those skilled in the art of preparing the various modifications. As used herein the term “ginseng” includes Panax ginseng, P. quiquefolius, P. notoginseng, P. japonicas, P. trifolium, P. pseudoginseng, P. vietnamensis, and Panax quinquefolium, but is not limited to these.

Used in the present invention, “red ginseng (Panax ginseng C.A. Meyer)” is prepared by steam or sun-dried, preferably manufactured by heating the fresh ginseng, more preferably the ginseng which has steamed for several hours at 98-100° C. and then well-dried at about 60° C.

Red ginseng is manufactured by the following conventional methods; washing, screening, steaming, drying, and removal of hair. A further drying (sun-drying) and steaming was undertaken followed by grade selection, weighing, pressing, drying, and progressing to packaging. Prepared samples were then cut, extracted between at 80 and 100° C., and concentrated at 60-90° C. (Red ginseng quality control for Jinan Red Ginseng January 2011, the Jinan Red Ginseng Research Institute).

The present invention includes conventional solvent extraction preferably by using (a) water, (b) anhydrous alcohol or alcohol having 1-4 carbon atoms such as methanol, ethanol, propanol, butanol, n-propanol, iso-propanol and n-butanol, etc, (c) mixture of water and alcohol from (b), (d) acetone, (e) ethyl acetate, (f) chloroform, (g) 1,3-butylene glycol, (h) n-hexane, (i) diethyl ether, or (j) butyl acetate. Preferred solvents are methanol, ethanol, butanol, and water, and the most preferred solvent is water.

The extract of the present invention is obtained by using the above-mentioned solvent, as well as those obtained by applying further refining processes. By additional processes such as distillation under reduced pressure, freeze-drying or spray-drying, the extract of the present invention can be manufactured in the form of powder.

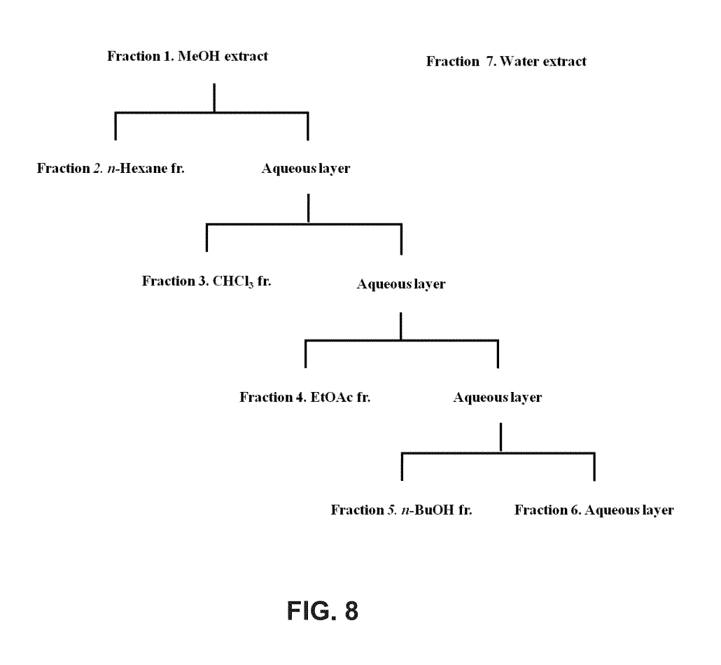

Ginseng extract improves the hydraulic conductivity of Bruch's membrane. Ginseng water extract (Fraction 7 in FIG. 8) and final aqueous layer (Fraction 6 in FIG. 8) were most effective to improve hydraulic conductivity, and methanol layer (Fraction 1 in FIG. 8) and butanol layer (Fraction 5 in FIG. 8) also showed effects on improving hydraulic conductivity of Bruch's.

To prepare ginseng/red ginseng extract and ginsenosides Rg1, Rb1 and compound K as a composition of the present invention using normal methods of manufacturing the tablets, capsules, injections, etc. To manufacture the tablets for prevention and treatment for macular degeneration, the tablets can be prepared using a 1:1 ratio of the active ingredient and the sum of lactose, microcrystalline cellulose, magnesium stearate as vehicles.

For medication, the herbal extract itself can be used, or powders, granules, capsules or injections can be manufactured as a mixture with pharmaceutically acceptable carriers, forming agents, and diluents. In addition, since ginseng has been used for ages as a medicinal edible material, there are no limits on the useable dosage. However, the dosage can be varied depending on the patients' absorption rate, weight, age, gender, health status, diet, time of administration, method of administration, rate of excretion, and the severity of the disease. In general, ginseng/red ginseng extract and ginsenosides Rg1, Rb1 and compound K should be used at about 0.1 to 100 mg per kg of body weight.

A basic dosage unit will be formulated incorporating the suggested formulation such that multiples can be prescribed depending on the frequency of use (i.e., tablets per day), the amount required dependent on the severity of the condition presented, parameters determined by the physician-in—charge for monitoring the specific needs of a given patient or individual.

According to another aspect of the present invention where ginseng is used for normal elderly or early AMD, the supplement (at lower dosage) can be provided as a food supplement rather than a prescribed medicine. This low dosage formulation for fod supplementation can incorporate ginseng/red ginseng extract, ginsenosides Rg1, Rb1 and compound K as active ingredients for the prevention of visual problems.

In the present invention, health food refers to food with body modulating functions such as prevention and treatment of disease, improved defence mechanism and immunity, recovery from illness, and anti-ageing. It should be harmless to the human body for long-term use.

Ginseng/red ginseng extract and ginsenosides Rg1, Rb1 and compound K can be manufactured by a variety of methods known in the field of food and pharmaceutical sciences in order to be used for the prevention and treatment of macular degeneration as described above. It can be used itself or may be prepared with acceptable carriers, forming agents, diluents, and also can be prepared by mixing other food in any kind of form that can be ingested orally. Preferably, it can be manufactured as a beverage, pill, granule, tablet or in capsular form.

The functional health food preparation of the present invention may include additional ingredients typically to the food manufacturing processes. For example, when beverages are manufactured, in addition to the extracts of the present invention, more than one additional component such as citric acid, liquid fructose, sugar, glucose, acetic acid, malic acid, fruit juice, etc. can be incorporated.

Amount of health functional food with an active ingredient for prevention and treatment of macular degeneration according to the present invention can be decided appropriately depending on the age, gender, weight, condition, and the symptoms of the disease. Preferably about 0.01 g to 10.0 g per day is recommended for adults and it will provide benefits for prevention of macular degeneration.

### EXAMPLES

The examples of the invention will now be described in more detail. These examples are intended to illustrate some of the aspects of the present invention but should not be construed as being limited to the overall scope of the present invention.

### Example 1

### Manufacturing of Red Ginseng and Red Ginseng Extract

Red ginseng is prepared using with following process. Fresh ginseng is washed and steamed at 94˜98° C., vapor pressure 3 kg/cm², pressure 1.5 kg/cm², then primary drying for 12-20 hours at 60˜70° C., and sundried until 15-18% moisture is retained. Ginseng is extracted by one of the solvents, such as water, alcohol, or a mixture of water and alcohol. Water is added to the ginseng (5-10 times w/w) and the primary extraction is processed for 12 hours at 30˜85° C. This step is repeated and the second extraction is processed for 3 hours at 30˜85° C. A further two extractions are carried out and finally, the extract is filtered to remove any debris and cooled to 10˜15° C., and then purified by centrifugation and concentration.

TABLE 1
Ginsenosides in 10% red ginseng extract

| | Content (mg/1 mL) | | | | | | | | | | |
|---|---|---|---|---|---|---|---|---|---|---|---|
| | Rg1 | Re | Rf | Rg2 + Rh1 | Rb1 | Rc | Rb2 | Rb3 | Rd | Rg3 | Total |
| Primary Samples | 0.09 | 0.13 | 0.10 | 0.16 | 0.57 | 0.22 | 0.21 | 0.04 | 0.09 | 0.18 | 1.80 |
| Secondary samples | 0.05 | 0.08 | 0.07 | 0.11 | 0.46 | 0.17 | 0.19 | 0.05 | 0.11 | 0.11 | 1.40 |

### Example 2

### Manufacture of Ginseng Extract

Ginseng is washed with clean water and then freeze-dried to obtain a moisture-free ginseng powder. This is the used for extraction.

\textcircled{1} Water Extraction

Water extraction is performed by adding ×10 volume of water to the ginseng powder and boiling. The solution is then evaporated with the aid of a reflux condenser for 6 hours. The whole procedure is repeated twice more followed by filtration. Than the water extract is freeze-dried.

\textcircled{2} MeOH Extract

MeOH (L) was added to the freeze-dried ginseng and extracted twice at 60° C. for 4 hours followed by filtration,

\textcircled{3} Fractions

MeOH extract was suspended in water (FIG. 8). Some volume of n-hexane was added and left shaking to obtain the hexane extract. The aqueous layer after hexane extraction was sequentially fractionated using an equal volume of CHCl₃, EtOAc and n-BuOH. Each fraction, n-Hexane, CHCl₃, EtOAc and n-BuOH fractions were concentrated under reduced pressure.

### Example 3

### Improvement of Hydraulic Conductivity of Bruch's Membrane with Different Ginseng Fractions

Ginseng was extracted with solvents described in FIG. 8.

Bruch's samples were mounted in open-type Ussing chambers and perfused with Tris-HCl buffer. Eluted samples (fluid coming through Bruch's membrane) were collected and measured for fluid transport. Control was perfused with Tris-HCl buffer whilst others were perfused with ginseng fractions for 24 hours.

([1] Moore D J, Hussain A A, Marshall J. (1995). Age-related variation in the hydraulic conductivity of Bruch's membrane. Invest. Ophthalmol. Vis. Sci. 36(7): 1290-7. [2] Starita C, Hussain A A, Pagliarini S, Marshall J. (1996) Hydrodynamics of ageing Bruch's membrane: implications for macular disease. Exp. Eye Res. 62(5): 565-72.)

Incubation of Bruch's membrane with the 0.5-2% of ginseng fractions for 24 hours improved the hydraulic conductivity (Table 2). Effect on hydraulic conductivity with individual fractions was tested. There was little, effect with the hexane layer (Fraction 2), the chloroform layer (Fraction 3), and the ethyl acetate layer (Fraction 4) in FIG. 8. There is some improvement in the methanol layer (Fraction 1) and the butanol layer (Fraction 5) in FIG. 8. Significant improvements in hydraulic conductivity of Bruch's were obtained using the aqueous layer extracted by hot water (Fraction 7, p<0.001) and the final aqueous layer (Fraction 6, p<0.01). These fractions and red ginseng extract showed significant improvement in hydraulic conductivity.

TABLE 2
Effect of Ginseng fractions on the hydraulic conductivity of human
Bruch's membrane.

| | | | | Fold change | |
|---|---|---|---|---|---|
| | Fraction | Fraction | | in hydraulic | |
| | Number | concentration | Number | conductivity | Significance |
| | in FIG. 8. | examined | of donors | over basal | level |
| Control | — | Incubated in | 18 | 1.1 ± 0.2 | — |
| | | Tris-HCl | | | |
| Whole Ginseng | — | 10% | 4 | 1.68 ± 0.16 | P < 0.001 |
| extract | | | | | |
| Ginseng | 5 | 0.5-2% | 7 | 1.20 ± 0.24 | NS |
| fraction 1 | | | | | |
| (butanol | | | | | |
| extract) | | | | | |
| Ginseng | 1 | 0.5-2% | 8 | 1.19 ± 0.08 | NS |
| fraction 2 | | | | | |
| (methanol | | | | | |
| extract) | | | | | |
| Ginseng | 7 | 0.5-2% | 13 | 1.46 ± 0.36 | P < 0.001 |
| fraction 3 | | | | | |
| (water extract) | | | | | |
| Ginseng | 6 | 1-2% | 9 | 1.43 ± 0.42 | P < 0.01 |
| fraction 4 | | | | | |
| (final extract) | | | | | |

### Example 4

### Effect of Ginsenosides Rg1, Rb1 and Compound K on Bruch's Membrane on the Hydraulic Conductivity of Human Bruch's Membrane

Transport was tested as described in Example 3, and measured after 24 hours incubation with ginsenosides.

The effect of incubation with Rg1, Rb1, and compound K on the hydraulic conductivity of human Bruch's membrane was assessed (Table 3). Compound K (190 ug/ml) increased hydraulic conductivity (p<0.005, Table 3). Similarly hydraulic conductivities of Bruch's were also increased by Rg1 (200 ug/ml) and Rb1 (200 ug/ml).

TABLE 3
Effect of Rg1, Rb1 and compound K on the hydraulic conductivity
of human Bruch's membrane.

| | | | Fold change | |
|---|---|---|---|---|
| | | | in | |
| | Fraction | | hydraulic | |
| | concentration | Number of | conductivity | Significance |
| | examined | donors | over basal | level |
| Control | Incubated in | 18 | 1.1 ± 0.2 | — |
| | Tris-HCl | | | |
| Compound K | 190 ug/ml | 5 | 1.45 ± 0.15 | P < 0.005 |
| Rg1 | 200 ug/ml | 1 | 1.2 | — |
| Rb1 | 200 ug/ml | 1 | 1.27 | — |

### Example 5

### Effect of Red Ginseng Extract on the Hydraulic Conductivity of Human Bruch's Membrane

Transport was tested as described in Example 3, and measured after 24 hours incubation with 10% red ginseng extract.

A 24-hour incubation of isolated Bruch's (17 donors, age range 12-87 years) with 10% red ginseng extract was associated with a 2.2-fold increase in the hydraulic conductivity of the membrane (FIG. 3c). The results of basal and post-red ginseng extract incubation are presented as a function of age of donor in the logarithmic plot of FIG. 3d. In the plot, the exponential decline in transport is indicated by the best-fit non-linear regression lines. KRG incubation displaced the curves upwards (as described in FIG. 3b) thereby increasing the age at which these curves meet the failure threshold. The hydraulic conductivities obtained after red ginseng extract treatment were those associated with donors 20-25 years younger. Thus red ginseng extract improves the hydraulic conductivity of Bruch's membrane.

Changes in hydraulic conductivity with commercially produced red ginseng extract (1, 2: Cheonjiyang, 3: Cheong-Kwan-Jang, 4: Keumsan Korean Ginseng Gold) were measured in the same way as above (FIG. 3e). All products improved hydraulic conductivity of the membrane. This means that all the different extraction methods for ginseng produce final products that improve the hydraulic conductivity of Bruch's membrane.

### Example 6

### Ginseng Mediated Improvement in the Diffusional Status of Human Bruch's Membrane

The diffusional status of Bruch's membrane was assessed with respect to the diffusion of FITC-albumin (MW 65 kDa) at a concentration gradient of 0.1 mM over 12 hours of incubation. Altogether, 44 donor samples of Bruch's (age-range 12-92 years) were mounted in Ussing chambers and basal diffusion rates determined. Then, 11 samples were incubated in Tris-HCL buffer and 33 in 10% ginseng for 24 hours. ([1] Hussain A A, Starita C, Hodgetts A, Marshall J. (2010) Macromolecular characteristics of ageing human Bruchmembrane: implications for age-related macular degeneration (AMD). Exp. Eye Res. 90:703-710) Incubation of Bruch's (33 donors, age range 12-92 years) increased the diffusional status of the membrane by 1.9-fold, p<0.001 (FIG. 4c). Basal diffusional levels showed an exponential decline with age. A semi-logarithmic plot of basal and post-KRG incubations as a function of age is shown in FIG. 4d. KRG incubations shifted the diffusional-age curves upwards towards improved diffusional status. KRG incubations improved diffusional rates to those associated with donors 16-21 years younger. Thus KRG improves the diffusional status of Bruch's membrane.

### Example 7

### KRG Mediated Removal of Bound and Trapped MMPs from Bruch's Membrane

Ageing of Bruch's is associated with entrapment and binding of proteinaceous material to the matrix of the membrane, contributing to the loss in transport properties. In the MMP Pathway described earlier (FIG. 2), high molecular weight MMP species exist largely in the bound form to the membrane. The process results in lowered levels of free MMPs available for the activation process. Lowered levels of activated MMPs are detrimental to the renewal and regeneration of Bruch's membrane.

Human Bruch's-choroid preparations were perfused for a period of 30 hours with Tris-HCl buffer to remove all free and mobile components of Bruch's membrane. In Tris-HCl eluted samples (T1-T4), most of the free MMPs were released in the first collection (T1) with amounts decreasing over the subsequent collections. T3 collections contained very little MMP activity. Subsequent elution with 10% ginseng resulted in the release of large amounts of bound HMW2, HMW1 and pro-MMP9 species together with active enzymes (FIG. 5a). In the case of MMPs, the release of activated species would serve to degrade other abnormal components helping to regenerate the membrane. KRG therefore, by releasing bound fractions from the matrix would contribute to improving the transport properties of the membrane.

### Example 8

### KRG Mediated Removal of Bound and Trapped Proteins from Bruch's Membrane

Ageing of Bruch's is associated with entrapment and binding of proteinaceous material to the matrix of the membrane, contributing to the loss in transport properties. As described in Example 7, Human Bruch's-choroid preparations were perfused for a period of 30 hours with Tris-HCl buffer to remove all free and mobile components of Bruch's membrane (FIG. 5b). The amount of proteins after 10% ginseng incubation was determined by Bradford assay. KRG therefore, by releasing bound fractions from the matrix would contribute to improving the transport properties of the membrane.

### Example 9

### Effects of KRG on the Release of MMPs from the Retinal Pigment Epithelium (RPE) of Pig Eyes

Pig whole eyecup preparations of RPE were used in these experiments. Standard buffer for incubations utilised Dulbecco's Minimal Essential Medium supplemented with foetal calf serum and antibiotics. Eyecups were incubated in this basal medium as control and in medium containing ginseng extract. Following a 6-24 hour incubation, the incubating medium was collected and subjected to gelatin zymography to analyse the type and level of MMP species present. In control medium, both pro- and active MMP 2&9 species are released continuously from the RPE (FIG. 7). Incubation with 10% Ginseng extract resulted in increased release of pro-MMP9 but decreased release of active MMP-9. On the other hand, the release of both pro- and active MMP2 species was elevated, and also activated HMW1. This effect of KRG is conducive for regenerating Bruch's membrane.

Thus, KRG increase the release of active MMP2 from RPE cells, a key enzyme involved in the regenerative process for improving the structural and functional aspects of Bruch's membrane.

### Example 10

### Effects of Ginsenosides Rg1, Rb1, and Compound K on the Release of MMPs from the Retinal Pigment Epithelium (RPE) of Pig Eyes

Experiments were performed using the methodology described in Example 9. Compound K consistently increased levels of Pro-MMPs 2&9 and active MMP2, again a very useful result for supplementing Ginseng extract with this compound (FIG. 7). Compound K activated HMW1. Rg1 increased the amount of pro-MMP9. Ginsenoside Rb1 was effective in reducing the level of pro-MMP9 but without effect on the level of active MMP9. Rb1 increased activated HMW1 levels. Rb1 however did increase levels of both pro- and active MMP2 species.

Thus, compound K and ginsenoside Rb1 increase the release of active MMP2, a key enzyme involved in the regenerative process for improving the structural and functional aspects of Bruch's membrane.

### Example 11

### Removal of Lipid Components from Bruch's Membrane after Treatment with Ginseng

The age-related accumulation of lipids and their likely effects on the transport pathways of Bruch's have been well documented. Initially, samples of human Bruch's-choroid were perfused with Tris-HCl buffer to remove the soluble components within the membrane. Half of the samples were further perfused with Tris-HCl and the other half with 10% Ginseng extract. Lipids were then extracted from the tissue samples and separated by thin layer chromatography on Silica Gel plates (FIG. 6).

Bruch's preparations were perfused with Tris-HCl for a period of 24 hours to remove free and mobile components of Bruch's membrane. Some of the samples were further perfused with Tris-HCl buffer whilst others were perfused with 10% ginseng for 24 hours. Samples were then removed from the chambers and lipids extracted with chloroform: methanol (2:1 vol). These samples were concentrated and applied to silica gel chromatographic plates and developed half-way up the plate in chloroform:methanol:acetic acid:water (50:30:8:3) with full development in heptane:diethyl ether:acetic acid (70:30:2). The chromatographs were stained for lipids with 0.2% amide black 10B. Samples perfused with Tris-HCl (T) showed the presence of cholesterol esters, triglycerides, cholesterol and two unidentified lipid components (arrow heads). Samples perfused with ginseng (G) did not show the presence of cholesterol, or the two unidentified lipid components. Thus, ginseng is capable of removing some of the lipid components of Bruch's membrane. This action of ginseng would assist in the regeneration of the membrane.

Following formulations are only examples for combinations of formulation and dosages, but the present invention is not limited thereto.

### 1. Formulation 1a

0.01-10 g ginseng/red ginseng extract

### 2. Formulation 1b

0-10 g ginseng/red ginseng extract+0-30 mg Rg1+0-30 mg Rb1+0-30 mg compound K

Normal maintenance of retinal function in the population aged 30-50 years. Whole ginseng extract at a dose of 1-2 g/day or with minimal supplementation with ginsenoside Rg1, Rb1 and compound K.

### 3. Formulation 2

0-10 g ginseng/red ginseng extract+0-50 mg Rg1+0-50 mg Rb1+0-50 mg compound K

Normal maintenance of retinal function in the population aged 50+ years. Whole ginseng extract at a dose of 1-3 g/day with increased supplementation with ginsenoside Rg1, Rb1 and compound K

### 4. Formulation 3

0-10 g ginseng/red ginseng extract+0-70 mg Rg1+0-70 mg Rb1+0-70 mg compound K

Functional improvement in people with high risk factors for AMD and for those diagnosed with early stages of macular disease. The purpose of this intervention is to delay or slow the progression of the disease. Whole ginseng extract at a dose of 2-4 g/day with increased supplementation with ginsenoside Rg1, Rb1 and compound K.

Pre-treatment for patients undergoing stem or RPE cell transplantation as a possible treatment for age-related macular degeneration. The viability of the transplanted cells would be much enhanced if the transportation pathways of underlying Bruch's were improved prior to transplant enabling better attachment and survival of transplanted cells. A regime with Formulation 3, 2-3 months prior to the transplant is suggested.

Pre-treatment of patients undergoing micro-nano pulse laser therapy for age-related macular degeneration. Formulation 3 for a period of 2-3 months would prime both Bruch's and the RPE since the laser procedures are dependent on a MMP response by the RPE for their mechanism of action.

### 5. Formulation 4

0-10 g ginseng/red ginseng extract+0-100 mg Rg1+0-100 mg Rb1+0-100 mg compound K+800 ug Vitamin A+5 ug Vitamin D+12 mg Vitamin E+80 mg Vitamin C+10 mg Zinc+1 mg Copper

Treatment for patients with age-related macular disease. Whole ginseng extract at a dose of 3-6 g/day supplemented with (a) high levels of ginsenoside Rg1+Rb1 and compound K and (b) vitamins and minerals at a much lower concentration than that prescribed by the AREDS study.

### Preparation Example 1

### Preparation of Tablets

Tablets for oral administration were prepared with following composition with ginseng or red ginseng extract or ginsenosides Rg1, Rb1 and compound K by wet or dry granulation method.

Composition: 200 mg, by selecting one or more of ginseng extract, ginseng extract, ginsenoside Rg1, Rb1 and compound K+10 mg light anhydrous silicic acid+2 mg of magnesium stearate+50 mg microcrystalline cellulose+25 mg sodium starch glycolate+101 mg of lactose+12 mg povidone+anhydrous ethanol.

### Preparation Example 2

### Preparation of Injections

Injections were prepared with following composition with ginseng or red ginseng extract or ginsenosides Rg1, Rb1 and compound K.

Composition: 200 mg with one or more of ingredient from ginseng/red ginseng extract, Rg1, Rb1 or compound K+180 mg mannitol+25 mg sodium phosphate+2974 mg Purified water for injections.

### Preparation Example 2

### Manufacture of Beverages

Beverages were prepared with following composition with ginseng or red ginseng extract or ginsenosides Rg1, Rb1 and compound K.

200 mg, by selecting one or more of ginseng extract, ginseng extract, ginsenoside Rg1, Rb1 and compound K+75 mg Vitamin C+4 mg Vitamin B2+3 mg Vitamin B6+1,000 mg dietary fiber+20,000 mg fructose syrup+100 mg emulsifier+50 mg flavoring in total volume of 50 ml with purified water.

The present invention shows that the composition of ginseng/red ginseng extract and ginsenosides Rg1, Rb1 and compound K as the active ingredient released bound or trapped proteins and MMPs from Bruch's membrane and also removed lipid components. The composition also increased the release of MMPs from RPE cells. Outstanding improvement of hydraulic conductivity and diffusion will help to (a) maintain and protect visual function in the normal population, (b) restore deficits in visual function in the elderly population, (c) slow and perhaps prevent the onset of AMD in those at high risk or in the early phase of maculopathy, and (d) slow or reverse the progression of the disease in AMD patients.

The retinal regenerative ginseng therapeutic protocol is a method for regenerating the functional properties of Bruch's membrane based on the pharmacological actions of Korean Red Ginseng (KRG). KRG improves transport functions by removing trapped material from the membrane and by inducing cellular responses from the retinal pigment epithelium (RPE) that facilitate the turnover of the matrix of Bruch's. The method essentially displaces the ageing degenerative functions such that functional failure can be avoided within the human life span. Depending on the dosage and supplementation status, retinal regenerative ginseng therapy is suitable both as a preventative and treatment procedure. Visual impairment in the elderly due to reduced transport of vitamin A, essential metals, and antioxidants across Bruch's can be prevented by oral administration of KRG alone. KRG supplementation with ginsenosides and trace elements would be suitable both as a prophylactic and treatment option to reduce or reverse the progression of AMD. The composition according to the present invention removes wastes from Bruch's membrane, releases the bound or trapped MMP species, and activates MMP enzymes to regenerate Bruch's membrane.

We claim:

1. A method for treating macular degeneration in a mammal administering to a mammal in need thereof, a therapeutically effective amount of a composition comprising one or more of ginseng extract, ginseng red extract, ginsenoside Rg1, ginsenoside Rb1 or ginsenoside compound K as an active ingredient.

2. The method according to claim 1, wherein the active ingredient activates MMP release from RPE cells.

3. The method according to claim 1, wherein the active ingredient regenerates, maintains, and improves function of Bruch's membrane.

4. (canceled)

5. (canceled)

6. The method according to claim 3, wherein the active ingredient assists in removing trapped and bound proteins from the matrix of Bruch's thereby allowing improved hydraulic and diffusional pathways through the membrane.

7. The method according to claim 3, wherein the active ingredient releases bound pro- and active MMPs 2 and 9 from the matrix of the membrane.

8. The method according to claim 3, wherein the active ingredient removes the age-accumulated lipid components of the debris deposited in Bruch's facilitating the structural and functional regeneration of the membrane.

9. The method according to claim 3, wherein the active ingredient stimulates the MMP system stimulating the release of active MMP2 from the RPE.

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. A method for treating macular degeneration in a mammal administering to a mammal in need thereof, a therapeutically effective amount of a composition comprising therapeutic one or more of ginseng extract, ginseng red extract, qinsenoside Rg1, ginsenoside Rb1 or ginsenoside compound K as an active ingredient before pre-treatment for patients undergoing stem or RPE cell transplantation.

26. A method for treating macular degeneration in a mammal administering to a mammal in need thereof, a therapeutically effective amount of a composition comprising one or more of ginseng extract, ginseng red extract, ginsenoside Rg1, ginsenoside Rb1 or ginsenoside compound K as an active ingredient before treatment of patients undergoing micro-nano pulse laser therapy for age-related macular degeneration.

27.-48. (canceled)
